SEQUENCE LISTING (23 sequences)

SEQ ID NO: 1 - DNA, 20 bp, Homo sapiens
agtctctcagctggtacacg

SEQ ID NO: 2 - DNA, 45 bp, Homo sapiens
cgtgatagtctctcagctggtacacgcaggtcagggtggccgttt

SEQ ID NO: 3 - DNA, 20 bp, Homo sapiens
gcaggtcagggtggccgttt

SEQ ID NO: 4 - DNA, 26 bp, Homo sapiens
atgataggatgacaggggtgacgcag

SEQ ID NO: 5 - DNA, 42 bp, Homo sapiens
atgataggatgacaggggtgacgcaggtcagggtggccgttt

SEQ ID NO: 6 - DNA, 20 bp, Homo sapiens
gcaggtcagggtggccgttt

SEQ ID NO: 7 - DNA, 28 bp, Homo sapiens
tacgtagagagccgcaggacgtgcactt

SEQ ID NO: 8 - DNA, 20 bp, Homo sapiens
gcaggtcagggtggccgttt

SEQ ID NO: 9 - DNA, 28 bp, Homo sapiens
tacgtagagagccgcaggacgtgcactt

SEQ ID NO: 10 - DNA, 42 bp, Homo sapiens
tacgtagagagccgcaggacgtgcacttcagctccactgggg

SEQ ID NO: 11 - DNA, 20 bp, Homo sapiens
gcacttcagctccactgggg

SEQ ID NO: 12 - DNA, 26 bp, Homo sapiens
atgataggatgacaggggtgacgcag

SEQ ID NO: 13 - DNA, 42 bp, Homo sapiens
atgataggatgacaggggtgacgcagtacgtggtgaagtcct

SEQ ID NO: 14 - DNA, 20 bp, Homo sapiens
gcagtacgtggtgaagtcct

SEQ ID NO: 15 - DNA, 26 bp, Homo sapiens
atgatattgatcttgaggtcaggggc

SEQ ID NO: 16 - DNA, 44 bp, Homo sapiens
atgatattgatcttgaggtcaggggcaggtcagggtggccgttt

SEQ ID NO: 17 - DNA, 20 bp, Homo sapiens
gcaggtcagggtggccgttt

SEQ ID NO: 18 - DNA, 28 bp, Homo sapiens
tacgtagagagccgcaggacgtgcactt

SEQ ID NO: 19 - DNA, 42 bp, Homo sapiens
tacgtagagagccgcaggacgtgcacctcagctccacagggg

SEQ ID NO: 20 - DNA, 20 bp, Homo sapiens
gcacctcagctccacagggg

SEQ ID NO: 21 - DNA, 26 bp, Homo sapiens
atgataatgcccaggtagtatggcgg

SEQ ID NO: 22 - DNA, 43 bp, Homo sapiens
atgataatgcccaggtagtatggcggcaggagcaaggcggtca

SEQ ID NO: 23 - DNA, 20 bp, Homo sapiens
cggcaggagcaaggcggtca

(19) United States
(12) Patent Application Publication
(10) Pub. No.: US 2022/0259653 A1
(43) Pub. Date: Aug. 18, 2022

(54) Dimer avoided multiplex polymerase chain reaction for amplification of multiple targets

(71) Applicant: iRepertoire, Inc., Huntsville, AL (US)

(72) Inventors: Wenjing Pan, Huntsville, AL (US); Miranda Byrne-steele, Madison, AL (US); Xiaohong Hou, Madison, AL (US); Brittany Brown, Madison, AL (US); Jian Han, Huntsville, AL (US)

(73) Assignee: iRepertoire, Inc., Huntsville, AL (US)

(21) Appl. No.: 17/300,937

(22) Filed: Dec. 15, 2021

Related U.S. Application Data
(63) Continuation of application No. 16/492,882, filed on Sep. 10, 2019, now abandoned.
(60) Provisional application No. 62/469,309, filed on Mar. 9, 2017.

(51) Int. Cl. C12Q 1/6883; C12Q 1/6848; C12Q 1/686; G01N 30/02

(52) U.S. Cl. CPC C12Q 1/6883; C12Q 1/6848; C12Q 1/686; C12Q 2600/16; C12Q 2531/113; C12Q 2537/143; G01N 30/02

(57) ABSTRACT

The present disclosure relates to methods for amplifying nucleic acids that avoid problems associated with primer-dimer formation. The present methods are referred to herein as dimer avoided multiplex polymerase chain reaction (dam-PCR). The methods disclosed herein generally comprise the steps of reverse transcribing at least one first strand of DNA, for example cDNA from an RNA sample, wherein each first strand of DNA incorporates a reverse common primer binding site; selecting each first strand of DNA; synthesizing at least one second strand of DNA from each of the at least one first strand of DNA, wherein each second strand of DNA incorporates a forward common primer binding site; selecting each second strand of cDNA; and amplifying the DNA strands using common primers. Alternatively, the method may be performed using a gDNA template. The methods described herein, due to the selection of DNA strands and removal of unused primers prior to amplification, avoid primer-dimer formation and allow for greater sensitivity and efficiency compared with conventional multiplex PCR methods.

### CROSS REFERENCE TO RELATED APPLICATIONS

This application claims priority to U.S. application Ser. No.: 16/492,882, entitled “Dimer Avoided Multiplex Polymerase Chain Reaction for Amplification of Multiple Targets,” and filed on Sep. 10, 2019, International Application No. PCT/US2018/021816, entitled “Dimer Avoided Multiplex Polymerase Chain Reaction for Amplification of Multiple Targets” and having an international filing date of Mar. 9, 2018, and U.S. Provisional Patent Application No. 62/469,309, entitled, “Dimer Avoided Multiplex Polymerase Chain Reaction for Amplification of Multiple Targets,” and filed on Mar. 9, 2017, which are incorporated herein by reference.

### RELATED ART

Polymerase chain reaction (PCR) has enabled the use of DNA amplification for a variety of uses, including molecular diagnostic testing. Multiplex PCR methods have been developed to amplify multiple nucleic acids within a sample to enable detection and identification of multiple target sequences. With multiplex PCR, multiple primers must be selected that will bind specifically to different target sequences to allow amplification of those sequences. The use of multiple primers, however, has proven problematic under conventional multiplex PCR methodologies, which require the optimization of primer sets, temperature conditions, and enzymes to satisfy the different conditions that may be required for different primers. Consequently, considerable planning and testing may be required to find compatible primer sets for conventional multiplex PCR methods.

The challenges associated with multiplex PCR are exacerbated by the formation of primer-dimers, which are often generated when multiple primers are used. The formation of primer-dimers may produce results where some target sequences amplify very efficiently, whereas others amplify very inefficiently or fail to amplify at all. This potential for uneven amplification also makes it difficult to impossible to accurately perform end-point quantitative analysis and requires considerable primer optimization to determine which primers may be suitably combined in a particular multiplex PCR assay. The problem of primer-dimers may persist even in methods wherein gene-specific primers are used to enrich targets sequences during an initial PCR cycling prior to further amplification using a common primer. Nonetheless, the current paradigm is that the selection of primers must be optimized to achieve the ideal performance of multiplex PCR [e.g., Canzar, et al. Bioinformatics, 2016, 1-3 (“Canzar”)].

Therefore, a need remains for a method that is capable of amplifying multiple target sequences using multiple primers while avoiding the negative effects of primer-dimer formation without requiring onerous and costly primer optimization.

### SUMMARY OF INVENTION

In one embodiment, the present disclosure relates to a method comprising the steps of: reverse transcribing at least one first strand of cDNA from mRNA containing at least one target sequence, using a reverse primer mix, forming a first strand cDNA; wherein the reverse primer mix contains at least one reverse primer configured to incorporate a reverse common primer binding site into each first strand of cDNA; selecting each first strand cDNA; synthesizing at least one second strand of cDNA from each of the at least one first strand of cDNA using a forward primer mix, forming at least one first strand:second strand complex; wherein the forward primer mix contains at least one forward primer, each forward primer configured to bind to a particular first strand of cDNA and to incorporate a forward common primer binding site into each second strand of cDNA; selecting each second strand of cDNA; selecting each first strand:second strand complex; amplifying the cDNA strands using a reverse common primer which binds to the at least one reverse common primer binding site and using a forward common primer which binds to the at least one forward common primer binding site; and selecting the amplified cDNA strands. In certain embodiments, the method further comprises the step of amplifying the amplified cDNA strands using a reverse common primer which binds to the at least one reverse common primer binding site and using a forward common primer which binds to the at least one forward common primer binding site. In certain embodiments of the method, the reverse primer mix comprises at least one reverse primer, wherein the at least one reverse primer comprises additional nucleotides which incorporate into each first cDNA strand as an identifying marker. In certain embodiments of the method, the forward primer mix comprises at least one forward primer, wherein the at least one forward primer comprises additional nucleotides which incorporate into each second cDNA strand as an identifying marker. In certain embodiments of the method, each selection comprises separation of cDNA strands from primer mix using magnetic beads. In certain embodiments of the method, each selection comprises separation of cDNA strands from primer mix by column purification. In certain embodiments of the method, each selection comprises enzymatic cleavage of primer mix. In certain embodiments, the first strand cDNA comprises a first strand cDNA:RNA complex. In certain embodiments, the present disclosure relates to a method of diagnosing the presence of a disease in a subject, said method comprising: providing a sample from the subject, the sample suspected of containing a disease agent, wherein the disease agent is characterized by a target sequence; performing the method described in this paragraph on the nucleic acids in the sample; sequencing the amplified DNA strands; and detecting a target sequence from the disease agent. In certain embodiments, the present disclosure relates to a method for producing an immune status profile for a subject, the method comprising: performing the method described in this paragraph on the nucleic acids from a sample of white blood cells from the subject; sequencing the amplified DNA strands; and identifying and quantifying one or more DNA sequences representing T-cell receptor, antibody, and MHC rearrangements to create an immune status profile of the subject. In certain embodiments, the mRNA is obtained from a single cell.

In certain embodiments, the present disclosure relates to a method comprising the steps of: synthesizing at least one first strand of DNA from genomic DNA containing at least one target sequence using a first primer mix, forming a first strand:DNA complex; wherein the first primer mix contains at least one first primer, each first primer is configured to bind to a particular target sequence and to incorporate a first common primer binding site into each first strand of DNA; selecting each first strand:DNA complex; synthesizing at least one second strand of DNA from each of the at least one first strand of DNA using a second primer mix, forming a first strand:second strand complex; wherein the second primer mix contains at least one second primer, each second primer configured to bind to a particular first strand of DNA and to incorporate a second common primer binding site into each second strand of DNA; selecting each first strand:second strand complex; amplifying the DNA strands using a first common primer which binds to the at least one first common primer binding site and using a second common primer which binds to the at least one second common primer binding site; and selecting the amplified DNA strands. In certain embodiments, the genomic DNA is obtained from a single cell. In certain embodiments, the present disclosure relates to a method of diagnosing the presence of a disease in a subject, said method comprising: providing a sample from the subject, the sample suspected of containing a disease agent, wherein the disease agent is characterized by a target sequence; isolating nucleic acids from the sample; performing the method described in this paragraph on the isolated nucleic acids; sequencing the amplified DNA strands; and detecting a target sequence from the disease agent. In certain embodiments, the present disclosure relates to a method for producing an immune status profile for a subject, the method comprising: performing the method described in this paragraph on the nucleic acids from a sample of white blood cells from the subject; sequencing the amplified DNA strands; and identifying and quantifying one or more DNA sequences representing T-cell receptor, antibody, and MHC rearrangements to create an immune status profile of the subject.

In certain embodiments of the method: the first primer mix is a reverse primer mix, each first primer is a reverse primer, each first common primer is a reverse common primer and each first common primer binding site is a reverse common primer binding site; and the second primer mix is a forward primer mix, each second primer is a forward primer, each second common primer is a forward common primer and each second common primer binding site is a forward common primer binding site. In certain embodiments of the method: the first primer mix is a forward primer mix, each first primer is a forward primer, each first common primer is a forward common primer and each first common primer binding site is a forward common primer binding site; and the second primer mix is a reverse primer mix, each second primer is a reverse primer, each second common primer is a reverse common primer and each second common primer binding site is a reverse common primer binding site. In certain embodiments of the method: the first primer mix is a primer mix comprising at least one forward and at least one reverse primer, each first primer is a forward or a reverse primer, each first common primer is a forward or reverse common primer and each first common primer binding site is a forward or reverse common primer binding site; and the second primer mix comprises at least one forward and at least one reverse primer, wherein no forward or reverse primer in the second primer mix is included in the first primer mix, each second common primer is a forward or reverse common primer and each second common primer binding site is a forward or reverse common primer binding site. In certain embodiments, the method further comprises the step of amplifying the amplified DNA strands using a reverse common primer which binds to the at least one reverse common primer binding site and using a forward common primer which binds to the at least one forward common primer binding site. In certain embodiments of the method, the first primer mix comprises primers that comprise additional nucleotides which incorporate into each first DNA strand as an identifying marker. In certain embodiments of the method, the second primer mix comprises primers that comprise additional nucleotides which incorporate into each second DNA strand as an identifying marker. In certain embodiments of the method, each selection comprises separation of DNA strands from primer mix using magnetic heads. In certain embodiments of the method, each selection comprises separation of DNA strands from primer mix by column purification. In certain embodiments of the method, each selection comprises enzymatic cleavage of primer mix.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIGS. 1A-1C illustrate the steps of an exemplary embodiment of the dam-PCR methodology using an RNA template. FIG. 1A depicts the step of single cycle first strand cDNA synthesis by reverse transcription (first strand tagging), the removal of unused reverse primer mix, and selection of the first strand cDNA. FIG. 1B depicts the step of single cycle second strand DNA synthesis (second strand tagging), the removal of unused forward primer mix, and the selection of first strand:second strand DNA duplex. FIG. 1C depicts the step of amplifying the targets using common primer and the optional step of repeating selection and amplification.

FIGS. 2A-2C illustrate the steps of an exemplary embodiment of the dam-PCR methodology using a genomic DNA (“gDNA”) template. FIG. 2A depicts the step of first strand DNA synthesis (first strand tagging) with the first primer mix, the removal of unused first primer mix, and selection of the first strand:DNA duplex. FIG. 2B depicts the step of second strand DNA synthesis and the removal of unused second primer mix. FIG. 2C depicts the step of amplifying the targets using common primer and the optional step of repeating selection and amplification.

FIGS. 3A and 3B illustrate the effects of primer-dimer formation on output. FIG. 3A is a gel demonstrating the prevalence of primer-dimer formation, using arm-PCR on single cells and the corresponding reduction in primary product band strength due to primer-dimer formation. Percentages on the gel indicate the percentage of sequencing reads wasted on sequencing residual primer-dimer products even after final library clean-up and were obtained by analyzing next generation sequencing data for each cell's data. Each individual lane represents a unique single cell after amplification with an arm-PCR multiplex mix covering the alpha and beta TCR locus and additional phenotypic markers. FIG. 3B is a table illustrating the potential for primer-dimer formation among pairs of primers in a mix which includes both forward and reverse primers during PCR and the reads wasted as a result of primer-dimer formation, which can account for as many as 31% of the reads that were intended for the samples.

FIGS. 4A and 4B illustrate the formation of primer-dimers with as little as one base pair overlap. FIG. 4A is a gel showing the intentional formation of primer-dimers resulting from performing arm-PCR under several cycling conditions, including RT-single cell protocol (scRT), no-RT single cell protocol (scPCR), and iR-10-10 protocol (iR1010) in the absence of template. Lanes 1-3 represent the primer-dimer formation of a single nucleotide overlap, which form between primers iTRAV_2 and iTRAC despite the protocol used. Lanes 4-6 represent the primer-dimer formation of four nucleotide overlap between primers iTRAV_2 and IL-17F-Ri-09, which form despite the protocol used. Lanes 6-9 represent the primer-dimer formation of a six nucleotide overlap between primers iTRAV_2 and BCL6Ri_MD_11, which form despite the protocol used. Lanes 10-12 represent the primer-dimer formation of a six nucleotide overlap between primers iTRAV_40 and BCL6Ri_MD_11, which form despite the protocol used. FIG. 4B illustrates the specific overlapping base pairs in certain primer-dimers. The rank of the primer-dimer product in the sequencing results of single cells amplified through arm-PCR demonstrated in FIGS. 3A and 3B is also provided, where “Top 1” represents the highest rank primer-dimer frequency in the NGS sequencing results, where “Top 2” the second most abundant primer-dimer frequency in the NGS sequencing results and so forth. FIG. 4B illustrates sequences represented by SEQ ID NO:1 through SEQ ID NO: 23. Specifically, for “Top 3”, “iTRAC” is SEQ ID NO:1, “iTRAV 2” is SEQ ID NO:3, and the primer-dimer product is SEQ ID NO: 2. For “Top 6” upper pairs, “IL-17F-Ri-09” is SEQ ID NO:4, “iTRAV 2” is SEQ ID NO:6, and the primer-dimer product is SEQ ID NO: 5. For “Top 6” lower pairs, “BCL6Ri MD 11” is SEQ ID NO:7 and “iTRAV 2” is SEQ ID NO:8. For “Top 2”, “BCL6Ri MD 11” is SEQ ID NO:9, “iTRAV 40” is SEQ ID NO: 11, and the primer-dimer product is SEQ ID NO: 10. For “Top 4”, “IL-17F-Ri-09” is SEQ ID NO: 12, “iTRAV 41” is SEQ ID NO: 14, and the primer-dimer product is SEQ ID NO: 13. For “Top 1”, “FOXP3-Ri-09” is SEQ ID NO:15, “iTRAV 2” is SEQ ID NO:17, and the primer-dimer product is SEQ ID NO:16. For “Top 5”, “BCL6Ri MD 11” is SEQ ID NO:18, “iTRAV 23” is SEQ ID NO:20, and the primer-dimer product is SEQ ID NO: 19. For “Top 7”, “CTLA-4-Ri-09” is SEQ ID NO:21, “CD8-Fi” is SEQ ID NO:23, and the primer-dimer product is SEQ ID NO: 22.

FIG. 5 is a gel demonstrating that a single pair of primers with high primer-dimer propensity can eliminate amplification of the desired product band. In lane 6 of depicted agarose gel, four primers to amplify the target IL-10 are included in a successful PCR. The addition of T-bet_Fi eliminates amplification of the band of interest as demonstrated in lane 7. Likewise, a multiplex primer mix covering four primers for T-bet and including IL-17A-Fo amplifies the target successfully as shown in lane 9. However, the addition of one disruptive primer, FoxP3Ri reverse inside primer eliminates amplification of the primary product band as shown in lane 10.

FIG. 6 is a gel demonstrating that adjusting annealing temperature does not remove the primer-dimer effect. Four different pairs of primers known to form primer-dimers were tested under several annealing temperatures ranging from 59.9° C. to 66° C. to remove the primer-dimer formation to no avail.

FIGS. 7A and 7B are gels illustrating the effect of primer-dimer formation with arm-PCR versus dam-PCR. FIG. 7A is a gel demonstrating that dam-PCR overcomes the effect of inhibitory primer-dimers using bead selection. Lanes 1-2 are arm-PCR controls. Lanes 3-4 are arm-PCR controls with the spike-in of a pair of primers known to cause dimerization. Lanes 5-6 are single cycle dam-PCR with no primer-dimer pair spike-in. Lanes 7-8 are single cycle dam-PCR with primer-dimer pair spike-in. Lanes 9-10 are dam-PCR with linear amplification with no primer-dimer pair spike-in. Lanes 11-12 are dam-PCR with linear amplification with primer-dimer pair spike-in. Lane 13 is a negative control. FIG. 7B is a gel demonstrating that dam-PCR overcomes the effect of inhibitory primer-dimers using transfer instead of bead selection after the first round of amplification with the common forward and reverse primer for dam-PCR or after the first round of RT-PCR for arm-PCR. Lanes 1-2 are arm-PCR controls. Lanes 3-4 are arm-PCR controls with the spike-in of a pair of primers known to cause dimerization. Lanes 5-6 are single cycle dam-PCR with no primer-dimer pair spike-in. Lanes 7-8 are single cycle dam-PCR with primer-dimer pair spike-in. Lanes 9-10 are dam-PCR with linear amplification with no primer-dimer pair spike-in. Lanes 11-12 are dam-PCR with linear amplification with primer-dimer pair spike-in. Lane 13 is a negative control.

FIG. 8 is a gel comparing single cell amplification using dam-PCR versus arm-PCR. Lanes 1-6 represent single cells amplified with dam-PCR, while lanes 7-14 represent single cells amplified with arm-PCR. The dam-PCR amplified cells have a similar endpoint intensity and lack primer-dimers, while the arm-PCR amplified cells demonstrate variation in end point PCR and show primer-dimer amplification.

FIG. 9 is a gel demonstrating the ability of the selection steps to remove unused primer. The lanes labelled “standard curve” were used to assess the amount of carryover of primer between first strand tagging and second strand tagging and between second strand tagging and amplification. Lanes 1 and 2 represent a magnetic bead selection step which is performed two times, and when compared to the standard curve, removes greater than 99.99% of unused primer. Lanes 3 and 4 represent a magnetic bead selection step which is performed one time, and when compared to the standard curve, removes greater than 99.9% of unused primer.

FIGS. 10A-10B show gels demonstrating dam-PCR with gDNA with varying multiplex primer mixes. FIG. 10A shows arm-PCR (Lanes 1-4) versus dam-PCR (Lanes 5-9) for the TCR beta locus amplification. FIG. 10B shows arm-PCR versus dam-PCR with the tumor multiplex panel at two input amounts of gDNA. Lanes 1-7 represent 200 ng gDNA input with arm-PCR performed for Lanes 1-3 and dam-PCR for Lanes 4-7. Lanes 8-14 represent 540 ng input with arm-PCR performed for Lanes 8-10 and dam-PCR for Lanes 11-14.

FIG. 11 is an illustration depicting normal multiplex PCR of gDNA with primers designed to cover two exons. This type of design introduces an overlap between the two amplicons to enable gene assembly during bioinformatic processing. Such a method produces non-target amplification due to the compatibility of the additional primers needed to create the overlap, resulting in a competing shorter PCR product.

FIG. 12 is an illustration depicting dam-PCR of gDNA and benefits associated with dam-PCR compared to normal multiplex PCR. In particular, during first cycle tagging, a set of primers can be used covering the larger exon product. After clean-up of first primer mix, the second primer mix includes inside primers that only interact with their respective first strand products. Since these primers are not involved in any amplification and are only used once during tagging, there is no competing shorter PCR product generated.

FIG. 13 is an agarose gel comparison of an arm-PCR multiplex PCR strategy and a dam-PCR strategy to cover a long gDNA gene target while including an overlapping portion, specifically covering a HLA target. An illustration is provided above the agarose gel for clarity to demonstrate the position of the primers referenced in the agarose gel. Lane 1 shows the amplification pattern when arm-PCR is used to amplify gene Target A only. Lane 2 shows the amplification pattern when arm-PCR is used to amplify gene Target B only. Lane 3 shows the amplification pattern of one potential off-target amplification from the interaction of T2 Forward and T1 Reverse due to efforts of generating an overlapping segment. Lane 4 shows the long amplification product of T1 forward primer and T2 Reverse primer. Lanes 5-6 show the arm-PCR amplification from the fully multiplexed mix. The result is largely dominated by the less desirable short off-target product. The gene targets for Target A alone (produced from T1 Forward and T1 Reverse), Target B alone (produced from T2 forward and T2 Reverse), and Target A and B together (produced from T1 Forward and T2 Reverse) are smeared and barely present due to the short products competing with the desired product for DNA polymerase activity. Lane 7 shows the amplification pattern when dam-PCR is used to amplify gene Target A only with T1 Reverse during first cycle tagging and T1 Forward during second cycle tagging. Lane 8 shows the amplification pattern when dam-PCR is used to amplify gene Target B only with T2 Reverse during first cycle tagging and T2 Forward during second cycle tagging. Lanes 9-10 show the results from the fully multiplexed dam-PCR strategy. In the first round of tagging, T1 Forward and T2 Reverse are used to generate the longer first strand products. In the second round of tagging, T1 Reverse and T2 Forward interact independently with the first strand products generated during the first strand tagging to synthesize the second strand. After second strand tagging, clean-up, and amplification with a pair of primers common to the first round targets, there is no competing short products, and the amplification of the desired products is achieved.

### DETAILED DESCRIPTION

The present disclosure relates to methods for amplifying nucleic acids that avoid problems associated with primer-dimer formation. The present methods are referred to herein as dimer avoided multiplex polymerase chain reaction (dam-PCR). The methods disclosed herein generally comprise the steps of reverse transcribing at least one first strand of DNA, for example cDNA from an RNA sample, wherein each first strand of DNA incorporates a reverse common primer binding site; selecting each first strand of DNA; synthesizing at least one second strand of DNA from each of the at least one first strand of DNA, wherein each second strand of cDNA incorporates a forward common primer binding site; selecting each second strand of cDNA; and amplifying the DNA strands using common primers. Alternatively, the method may be performed using a gDNA template. The methods described herein, due to the selection of DNA strands and removal of primers prior to amplification, avoid primer-dimer formation and allow for greater sensitivity and efficiency compared with conventional multiplex PCR methods.

As used herein, “disease” means an infection, symptom, or condition caused by or related to the agent.

As used herein, a “disease agent” means any organism, regardless of form, including, but not limited to a bacterium, a cancer cell, a virus, or a parasite that incorporates a nucleic acid sequence and causes or contributes to a disease in a subject.

As used herein, “first primer mix” means a mixture comprising at least one reverse primer and/or at least one forward primer configured to bind to gDNA.

As used herein, “forward primer mix” means a mixture comprising at least one forward primer.

As used herein, an “identifying marker” means a nucleotide sequence used as a label to identify the particular sample, nucleic acid strand or single cell source for mRNA or gDNA.

As used herein, “reverse primer mix” means a mixture comprising at least one reverse primer.

As used herein, “sample” means material comprising DNA or RNA.

As used herein, “second primer mix” means a mixture comprising at least one reverse primer and/or at least one forward primer configured to bind to at least one first strand of DNA.

As used herein, “subject” means a mammal, preferably a human.

Conventional multiplex PCR methods include a selection step (i.e., the removal of primer mix or separation of the DNA strands), if at all, only after amplicons are produced from PCR (e.g., arm-PCR [WO/2009/124293], tem-PCR [WO/2005/038039], each of which further requires the use of nested primers). If selection is performed after completion of PCR, however, Applicants show that primer-dimers will not only have the opportunity to form in the first few PCR cycles, the primer-dimers will also reduce the sensitivity of the reaction, because the primer-dimers will be continually competing with the targeted sequence for DNA polymerase activity during the PCR reaction. Further, the amplicons and primer-dimers will be difficult to separate due to similarity in molecular weight and charge. As demonstrated in the Examples below, in extreme cases amplification of primer-dimers dominate the reaction, completely eliminating amplification of the target sequence. DNA polymerases are more proficient at producing and binding to shorter amplicons than they are at producing longer products, thereby further exacerbating the creation of primer-dimers. In cases where primer-dimers are formed, but do not completely inhibit amplification of the target sequence, the desired amplicon amount is still reduced due to the competition between the target sequence and the primer-dimers, reducing the overall sensitivity of the reaction and creating products which, when carried over to sequencing, result in lost sequencing reads and unnecessary expense.

Applicants assert that the current paradigm of multiplex optimization is inherently flawed, because primer-dimers can form with as little as 1 bp overlap. Therefore, predicting primer-dimers is not possible and must be addressed using a new methodology. Here, Applicants show that the efficiency of separating the desired DNA strands from primer mix (referred to herein as “selection”) is much better when the selection is performed after reverse transcription instead of after PCR. If using relatively long oligonucleotides, such as when making next generation sequencing (NGS)-compatible libraries, primer-dimer formation can produce products approximately 200 bp in length, making separation from the desired product band much more difficult and less efficient. Placing the selection step after reverse transcription, however, implies the separation of an approximately 2 kb RNA:DNA duplex from individual primers (typically <80 bp), which is a much simpler separation to achieve. The additional selection after second strand synthesis in the described method is also easier than selection after PCR, because no primer-dimers have formed, and thus, the separation is between a first strand DNA:DNA duplex and shorter primers (<80 bp). In summary, sensitivity, which is critical to single cell applications, is already lost if waiting until after PCR to separate or rescue amplicons from primer-dimers. In contrast, by preventing the reverse primer mix from interacting with the forward primer mix (e.g., separation of reverse transcription and second strand cDNA synthesis) or by preventing the first primer mix from interacting with the second primer mix as with gDNA and by removing the reverse primers or forward primer mix (or removing the first primer mix or second primer mix as used with gDNA) prior to any PCR step, the possibility of primer-dimer formation is eliminated, and the energy of the reaction is focused on amplification of the desired product.

As additional support, Applicants assert that single cells may be considered a dynamic template with varying gene expression patterns. With single cells, it is not possible to predict how a multiplex system will respond, given the presence or absence of templates amounts that vary. Primer-dimers, that otherwise would not form in the presence of template, may form if a certain gene is absent, making primer design impossible due to the variation of expression at single cell level.

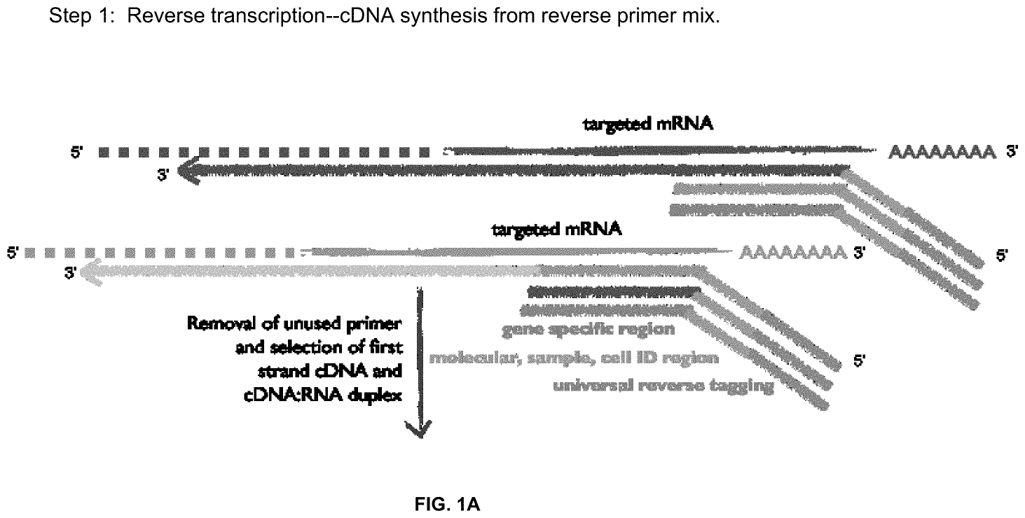

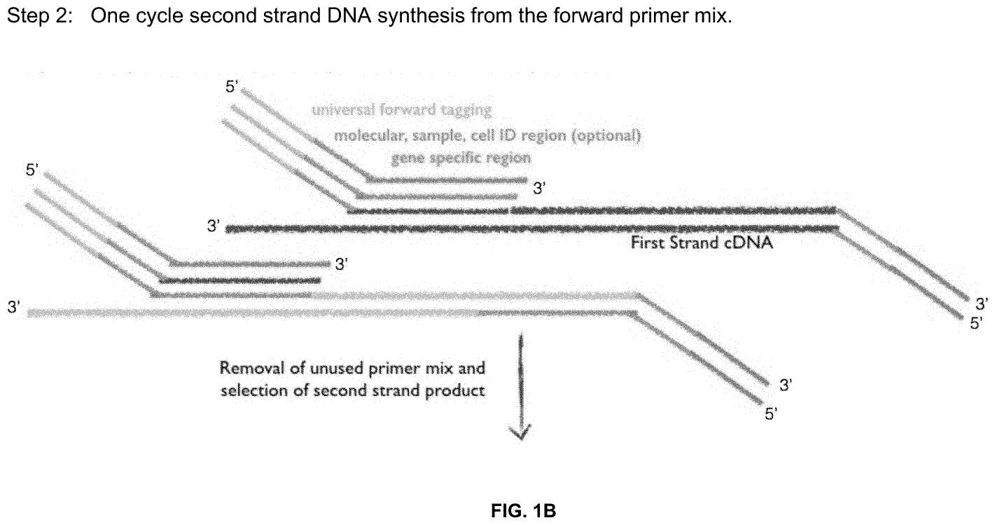

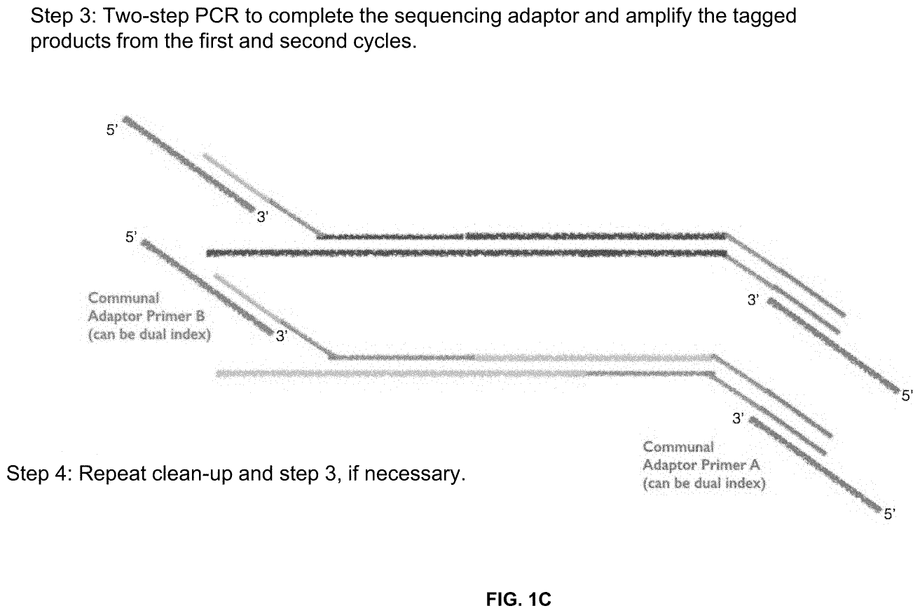

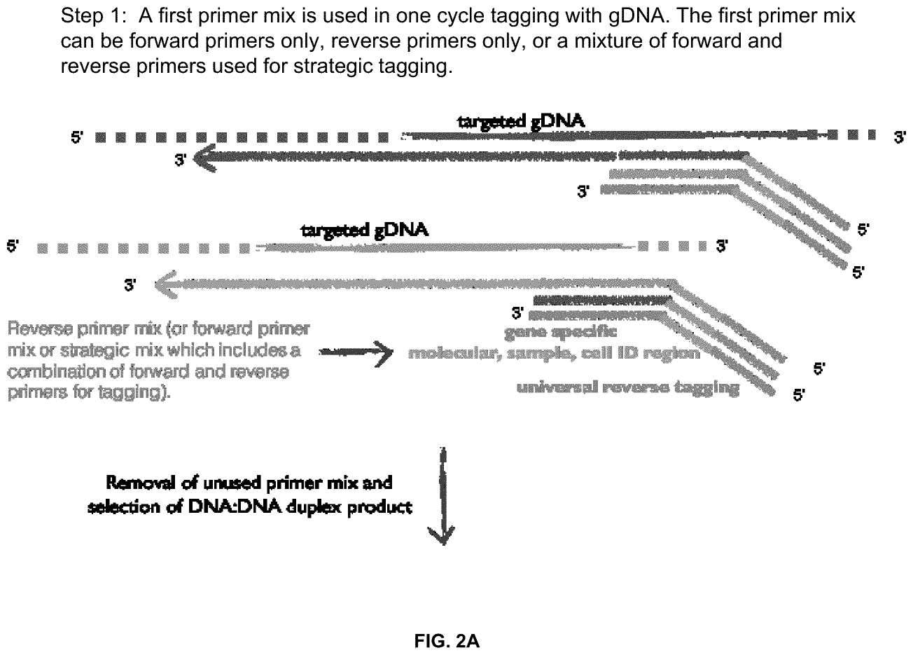

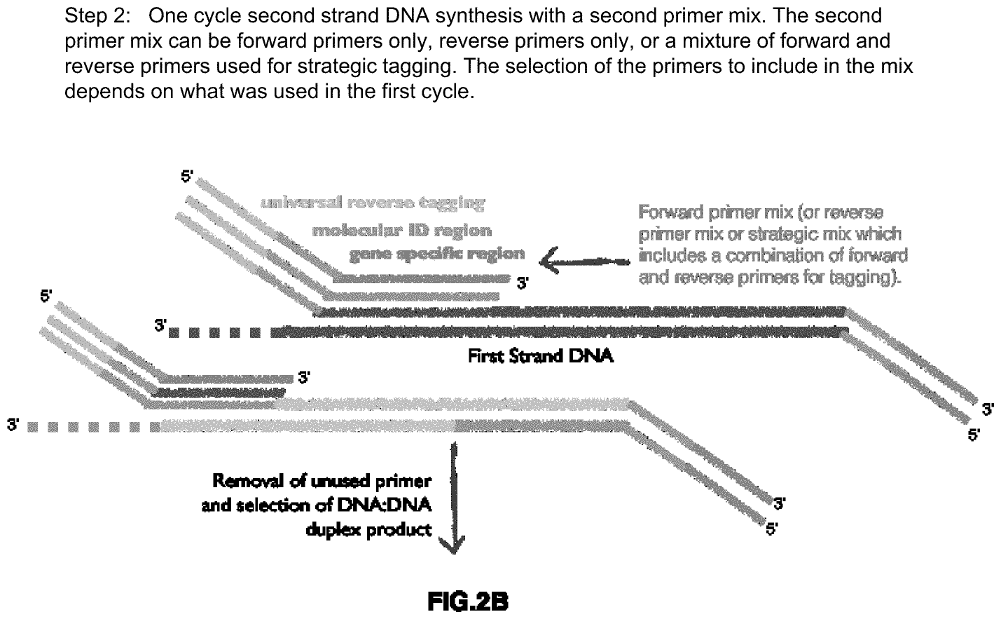

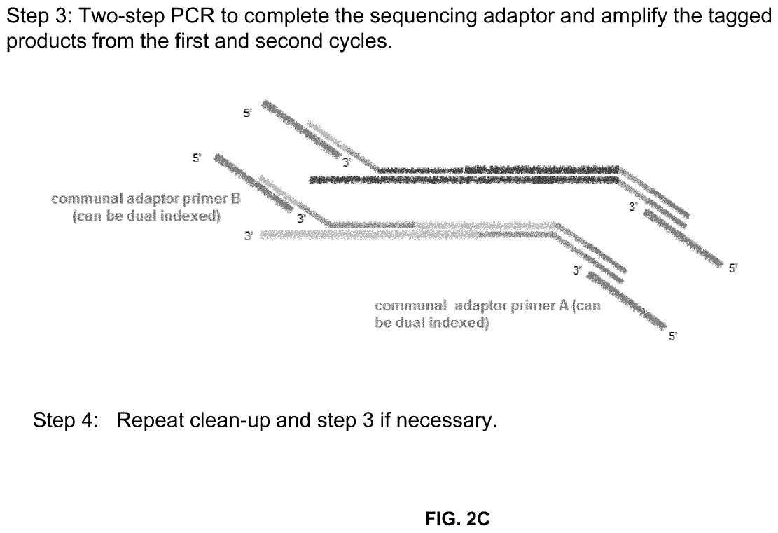

To overcome these difficulties, Applicants have developed a methodology in which primer-dimers are avoided in what is referred to herein as dimer avoided multiplex polymerase chain reaction (dam-PCR), which is a multiple-step multiplex reverse transcription (RT) PCR for RNA or a multiple-step multiplex PCR for gDNA. The reverse transcription step is performed separately from second strand DNA synthesis and from PCR amplification using a universal primer. Likewise for gDNA, strand tagging is performed by a first primer mix separately from second strand tagging which is performed with a second primer mix. By selecting the synthesized DNA at each step and tagging first strand cDNA or gDNA with one cycle of tagging and extension, the propensity for primer-dimer formation is avoided, and the DNA polymerase activity is focused on amplifying the targets of interest, rather than the primer-dimers, greatly increasing amplification sensitivity. An exemplary embodiment of the disclosed method using an RNA template is shown in FIGS. 1A-1C and an exemplary embodiment of the disclosed method using a gDNA template is shown in FIGS. 2A-2C.

During reverse transcription, one or more reverse primers containing both a 3′ gene specific and 5′ universal binding site are used for first strand cDNA synthesis. After reverse transcription, one key to primer-dimer mitigation is very accurate selection of the first strand cDNA:RNA complex (“first strand:RNA complex”) and efficient removal of oligonucleotides of sequence length (typically <80 bp) from this primary product band. This is particularly important if quantifying RNA by labeling each RNA with distinct oligonucleotides, as carryover of uniquely labelled primers will compromise the ability to quantify labelled nucleic acid species accurately. After reverse transcription, a selection step is performed to remove unused reverse primer. The “selection” step separates DNA strands (e.g., a first cDNA strand:RNA complex or single stranded first strand cDNA in solution) from unused primer mixes and can be magnetic bead-based (for example, solid-phase reversible immobilization (SPRI) beads), streptavidin-biotin bead-based, enzymatic, column-based, by gel purification, or other physical, chemical, or biochemical means to either actively select the DNA strands or, conversely, to remove the unused primer and any DNA polymerase. In the Examples, Applicants demonstrate a selection efficiency of removal of more than 99.99% of unused primer.

Once first strand cDNA synthesis is complete and the reverse transcription primers are removed efficiently by selection of the first strand cDNA, second strand cDNA synthesis is performed using a forward primer mix, termed second strand DNA “tagging”. One cycle of DNA polymerase activation followed by annealing is sufficient to tag the first strand cDNA products with the forward primer mix in absence of any reverse primer. Tags for individual nucleic acid species can also be introduced at this step since only one cycle is performed and unused primer is removed prior to PCR. It is also possible to perform limited cycles of linear amplification at this point (primer annealing and extension and/or isothermal amplification in the absence of reverse primer) to increase yield. However, too many additional cycles of annealing and extension may increase the risk of forming deleterious primer-dimers between primers in the forward-mix and will likely need to be empirically determined for a given multiplex system. Applicants demonstrate that a single cycle for first and second strand tagging, respectively, is sufficient to achieve amplification as is evidenced by amplification from single cells. After second-strand DNA synthesis, a second selection step is performed to remove unused forward primer mix. The selection step separates DNA strands from unused primer mixes and can be magnetic bead-based (for example, SPRI beads), streptavidin-biotin bead-based, enzymatic, column-based, by gel purification, or other physical, chemical, or biochemical means to either actively select the DNA strands or, conversely, to remove the unused primer and any DNA polymerase. The underlying concept is the same as the original selection, which is to remove the primer complexity so that primer-dimers do not form which can out-compete the primary and desired product.

At this point, the “tagged” DNA contains universal primer binding sites on both the 5′ and 3′ ends. All reverse primer and forward primer tagging mix is removed by this stage. The universal sites are generally the sequencing adaptors or a portion of the adaptors required for the NGS platform of choice. However, any universal engineered site would be applicable. PCR is performed using a pair of primers common to the universally “tagged” second strand cDNA. These primers complete the exponential stage of amplification. With primer complexity reduced to a two-primer system (in which the 3′ ends are not reverse complementary and in which the primer pair has been screened against primer-dimer formation), the propensity for primer-dimer production during this PCR is eliminated.

In cases of low input, such as single cell, it may be necessary to perform a second round of PCR. In this case, selection of the first round of PCR is completed as aforesaid, and a two-step PCR with the universal primers pairs is performed with fresh enzyme, buffer, and dNTP to enrich the first round products. In all cases, a final library selection is performed, and the libraries are directly ready for pooling, pre-sequencing QC, and sequencing using techniques known in the art.

Modifications for gDNA can be made; however, the same overall principle applies, avoidance of the generation of primer-dimers. The first round of PCR is performed with one set of the multiplexed primers only (first primer mix) using a single cycle denaturation and annealing step. To increase sensitivity, a linear amplification or isothermal amplification can be performed so long as primer-dimer formation among the mix is restricted. The first strand:DNA complex is purified as aforesaid, and the second strand synthesis is performed in a single cycle using the second primer mix. To increase sensitivity, a linear amplification or isothermal amplification can be performed. After second strand synthesis, selection is performed as aforesaid, and PCR is performed with a pair of universal primers, as described during the RNA process.

The methods disclosed herein may be used to detect the presence, and relative amounts present, of nucleic acids from viruses, bacteria, fungi, plant and/or animal cells for the evaluation of medical, environmental, food, and other samples to identify microorganisms and other agents within those samples.

One advantage of the methods described herein is that, unlike conventional multiplex PCR methods, primer-dimer formation is avoided, which greatly simplifies the selection of primer sets that otherwise would produce primer-dimers capable of impacting the amplification of a target sequence. Another advantage of the methods described herein is that the avoidance of primer-dimer formation results in highly sensitive multiplex PCR down to single cells. Using the disclosed methods, RNA may be targeted at a single cell level. Further, by reducing the number of wasted reads that would otherwise occur due to the presence of primer-dimers, the cost per read for each target sequence is significantly reduced. Another advantage to the method, particularly related to coverage of large gene segments with gDNA, is that data from overlapping amplicons can be made without detrimentally introducing shorter amplicon side-products that reduce the sensitivity. These side products are unavoidable if using a similar strategy with regular multiplex PCR. The dam-PCR method allows for strategic design of first and second primer mixes, allowing for longer coverage while restricting interaction between certain primers that produce short amplicon products that compete for DNA polymerase activity.

### EXAMPLES

### Material and Methods: dam-PCR and arm-PCR Set-Up

Primers covering the T-cell receptor alpha locus, beta locus, and additional phenotypic markers were multiplexed in the same mix or into forward and reverse mixes depending on the PCR strategy. The arm-PCR mixes consisted of 218 forward primers and 68 reverse primers in the same mix, covering 247 or more targets. Targets are defined in terms of reference sequences, but due to the variability of the rearranged TCR loci, the actual number of target sequences in a given sample is typically in the thousands for a bulk RNA sample. For a single cell, anywhere from 5-10 or more targets may be present depending on cell phenotype. For dam-PCR, the forward mixes were treated separately from the reverse mixes, and the outside primers associated with the nested portion of arm-PCR were excluded, for a total of 107 forward primers and 32 reverse primers. The inside primers, which contained the universal tag, were common to both mixes and included primer-pairs known to cause primer-dimer formation. In both primer sets, a portion of the Illumina dual-indexed compatible sequencing primer B was linked to each forward inside primer, while a portion of Illumina dual-indexed sequencing communal primer A and a sample barcode sequence of 6 nucleotides were linked to the reverse inside primers. In certain experiments, indices of 20 random nucleotides to tag individual nucleic acid species were included adjacent to the sample barcode.

For the arm-PCR approach, cDNA was reverse transcribed from a total RNA sample using the nested primer-mix of 286 primers and reagents from the OneStep RT-PCR kit (Qiagen, Valencia, Calif.). For arm-PCR, the first round of RT-PCR (termed “RT-PCR1” or “PCR1”) was performed at: 50° C., 60 minutes; 95° C., 15 minutes; 94° C., 30 seconds, 60° C., 5 minutes, 72° C., 30 seconds, for 10 cycles; 94° C., 30 seconds, 72° C., 3 minutes, for 10 cycles; 72° C., 5 minutes, and a hold of 4° C. A 0.7x SPRISelect bead selection (Beckman Coulter, Brea, Calif.) was performed after RT-PCR1, and nucleic acids products were eluted from the bead using the Promega Gotaq G2 Hotstart PCR mix (Promega, Madison, Wis.). A second round of PCR (termed “PCR2”) was performed with a set of communal primers that complete the Illumina adaptor sequences as: 95° C., 3 minutes; 94° C., 30 seconds, 72° C., 90 seconds, for 30 cycles; 72° C., 5 minutes and a hold of 4° C.

For dam-PCR, the reverse transcription step was performed in the presence of the reverse primer mix at 50° C. for 240 minutes using the Qiagen OneStep RT-PCR kit, and first strand cDNA was separated from the reverse primer mix by performing a 0.7x SPRI bead selection twice. After reverse transcription, second strand DNA synthesis was performed using Promega GoTaq G2 HotStart DNA polymerase in a Biorad C1000 thermocycler with the forward primer mix only (no reverse primer) in either one-cycle of tagging: 95° C., 3 minutes initial denaturation and hot start; 60-65° C., annealing, 3 minutes per degree change; and 72° C., 10 minutes extension with a final hold of 4° C.; or a linear amplification strategy: initial denaturation and hot start 95° C., 3 minutes, followed by 6-cycles of annealing and extension; 60° C., 5 minutes annealing, 72° C., 1 minute extension and a final hold of 4° C. After second strand DNA synthesis, the second strand DNA:DNA duplex was separated from the forward primer mix using a 0.7x SPRI bead selection twice. DNA products were eluted from the bead using the Promega Gotaq G2 Hotstart PCR mix, which contains one pair of primers common for the partial adaptor sequences introduced during the reverse and forward priming steps. Twenty-cycles of PCR equivalent to the total cycles of arm-PCR approach were used to amplify the cDNA with the universal primer pair: 95° C., 3 minutes; 94° C., 30 seconds, 72° C., 6 minutes, for 10 cycles; 94° C., 30 seconds, 72° C., 3 minutes, for 10 cycles; 72° C., 5 minutes, and a hold of 4° C. For dam-PCR, an additional SPRI bead selection was performed as previously described, and PCR was performed for a second time in the presence of fresh enzyme, buffer, and dNTP: 95° C., 3 minutes; 94° C., 30 seconds, 72° C., 90 seconds, for 30 cycles; 72° C., 5 minutes and a hold of 4° C.

After the second PCR reaction (i.e., PCR2 in the case of arm-PCR), 10 μL of the PCR product was run on a 2.5% agarose gel to assess amplification success. A 0.7x SPRI bead selection was used to select the libraries prior to sequencing for both arm-PCR and dam-PCR products. The final libraries were eluted from the beads in 25 μL of nuclease-free water and measured with a Nanodrop (Thermoscientific, Carlsbad, Calif.). Equimolar quantities of each library were pooled for sequencing, with the exception of the libraries generated with the primer-dimer spike-in. These libraries were pooled with half as much due to the availability of library. The pooled library was quantified with Qubit quantification and assessed with a Bioanalyzer. The library was then quantified with Kappa qPCR, diluted to 8 pM with a 10% PhiX spike-in, and sequenced with an IIlumina MiSeq v2, 500 cycle kit using 250 paired-end reads. An analogous strategy was used for described gDNA templates with the exception of the reverse transcription step which was removed. Strategic primer design was used when designing the first and second primer mix as described in the discussion to avoid production of short products.

### Effect of Primer-Dimer Formation on NGS Output: Leads to Loss of Sensitivity to the Genes of Interest, Wasted Reads, and thus, Increased Sequencing Expense

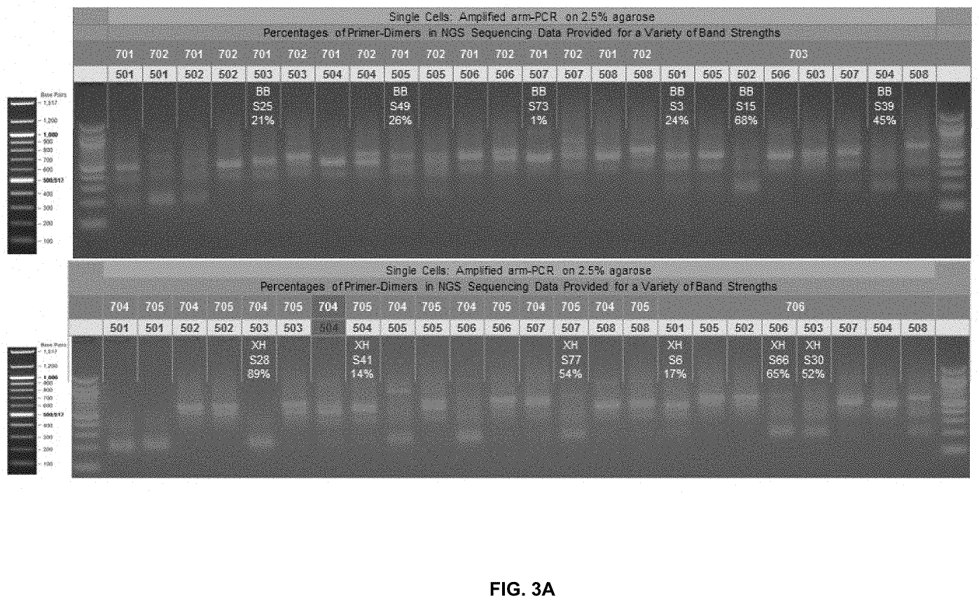

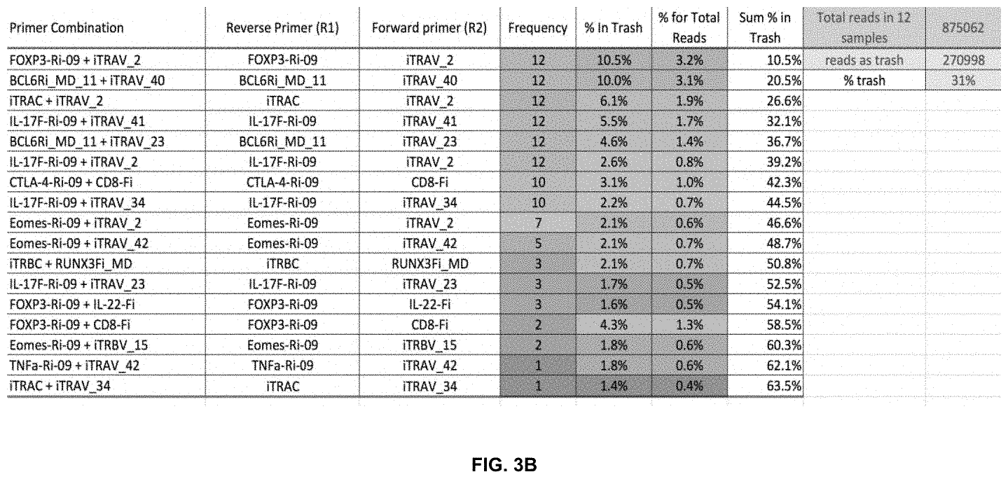

Traditional arm-PCR was used with the optimized multiplex mix to amplify single cells as demonstrated in the agarose gel in FIG. 3A. Single cells may be isolated using techniques known in the art. arm-PCR is a nested, multiplex RT PCR in which products are “rescued”, for example a small sampling from a completed first amplification reaction may be taken to provide amplicons for a second amplification, after RT-PCR.

After amplification, libraries representing each cell were pooled and subjected to an additional round of selection prior to sequencing on an Illumina MiSeq v2 500 cycle kit using 250 paired-end reads. Raw data were analyzed for evidence of primer-dimer relative to successful target reads. The percentage of reads occupied by primer-dimer sequences was found to be inversely proportional to band strength of the primary product band (FIG. 3B). For instance, for sample BB-S73, the primary product band was strong and only 1% of the sequencing reads for this cell were occupied by primer-dimers, whereas for sample XH-S28, there were relatively few product sequencing reads and 89% of this cell's data were dominated by primer-dimers. Even data for relatively strong products bands such as sample BB-S25 were occupied by 25% primer-dimers.

A benefit of targeted sequencing approaches when compared to other methods such as RNAseq is that less sequencing depth is required to cover genes of interest, because the genes of interest are specifically targeted and amplified. When single cells are analyzed, the costs can become exorbitant very quickly if 25-fold more reads are required to achieve the same coverage as a targeted-seq approach, particularly when each single cell is treated as a sample. Analysis of the sequences of the primer-dimers in the described experiment reveal that as much as 31% of the overall sequencing reads are occupied by primer-dimers, wasting valuable sequencing resources, resulting in undue cost in addition to loss of sensitivity in covering the genes of interest for a given cell. However, if amplification can be achieved equivalent to the strongest amplicon band, the primer-dimer waste is essentially eliminated while providing the benefits of reducing sequencing costs and increasing the sensitivity and coverage of the genes of interest.

### NGS of Primer-Dimers Reveals 1 BP Overlap is Sufficient to Form Primer-Dimers, Making Removal by Design Alone Impossible

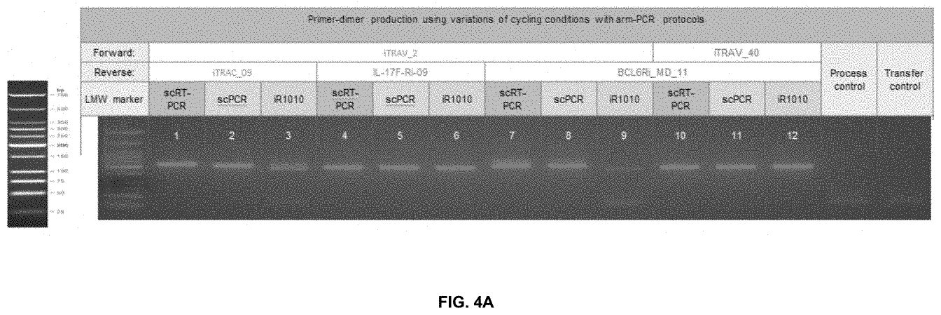

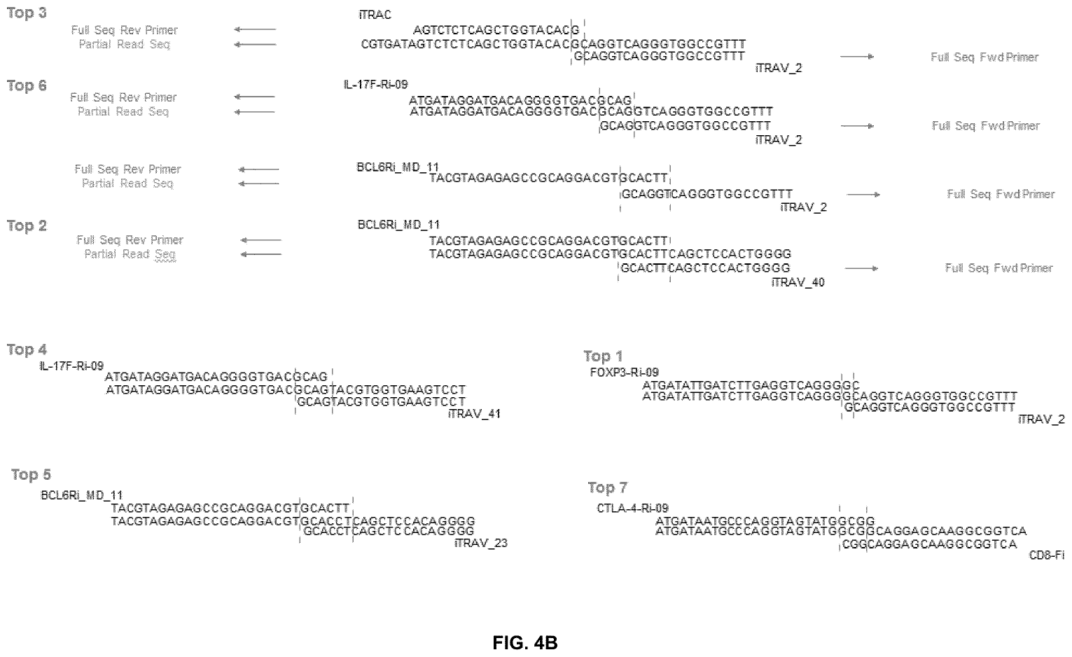

In an effort to remove primer-dimers, Applicants purposefully generated primer-dimers by performing arm-PCR with a multiplex mix in the absence of template (FIG. 4A) and sequenced the resulting amplicons using next generation sequencing (NGS) technology to highlight the primer sequences which might need to be redesigned. Surprisingly, more than a few hundred different interacting pairs were evident in the data with as few as 1 bp overlap yielding a dimerized product (FIG. 4B). In fact, the 1 bp overlap primer-dimer was one of the most frequently observed primer-dimer pairs. Some of the potential pairs were predictable based on base-pair complementarity and the 3′ end of each primer, but a 1 bp overlap is impossible to predict and thus is impossible to design around, again showing the fallacy of the current paradigm of “optimizing” primers for multiplex PCR (e.g., Canzar).

Cloning and sequencing individual clones of primer-dimers, however, does not provide the power to see the full extent of the issue and the sheer number of possible interactions. It was not evident prior to physically observing the sequence of the primer-dimer band with NGS that it is impossible to “out-design” primer-dimers. The previous result (FIG. 3A) demonstrates that even in a “successful” multiplex PCR, there is still significant primer-dimer production. This primer-dimer product competes throughout the entire PCR with the band of interest, greatly reducing the sensitivity of the PCR and compromising the coverage of the genes of interest for the sample. Prior to NGS, observation of the extent of the primer-dimer effect was unobtainable. Taken together, both of these results reveal that an alternate approach to PCR, using dam-PCR as disclosed herein, is the only possible solution to solve the issue of compatibility and sensitivity in multiplex PCR.

### Evidence of the Damage a Single Primer Pair Can Do to Multiplex PCR

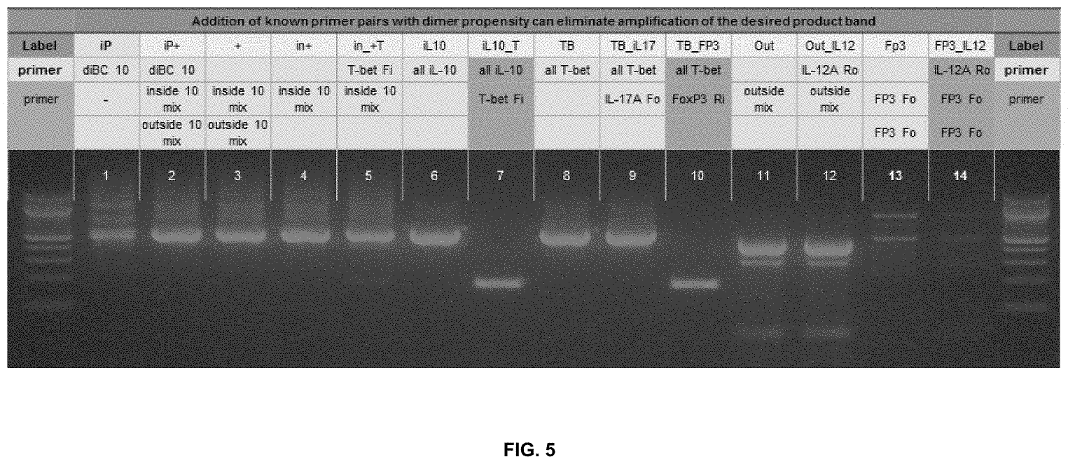

From FIG. 3A, described above, it is evident that primer-dimer formation can have an effect on sequencing yield, even when amplification is considered relatively “successful.” In other words, amplification yielded a band of interest, but the sequencing results demonstrated a significant amount of primer-dimer together with the product of interest. However, Applicants can also demonstrate in an extreme case an example of a primer-pair that eradicates amplification of the desired product band in a multiplex PCR mix. As demonstrated in FIG. 5, the addition of T-bet forward primer to a mix containing the primers for IL-10 resulted in the elimination of the desired product band which was present before the addition of this single primer to the mix. Similarly, the addition of FoxP3 reverse primer to a mix of T-bet forward and reverse primers similarly resulted in the loss of amplification of the primary product band.

Applicants demonstrated that a single primer pair could eliminate amplification of the targets of interest. In light of the present disclosure, which shows that the primer-dimers compete with the desired product for DNA polymerase activity during PCR, the many multiplex PCR failures may be considered “successful” (albeit undesired) primer-dimer amplification products, representing a paradigm shift in the understanding of multiplex PCR. Once that point is apparent, this disclosure demonstrates that the best PCR approach is to avoid the competition between the primary product band and potential primer-dimers for DNA polymerase activity, as is accomplished with the presently disclosed dam-PCR methodology.

### Raising Annealing Temperature is Insufficient to Overcome Primer-Dimer Propensity

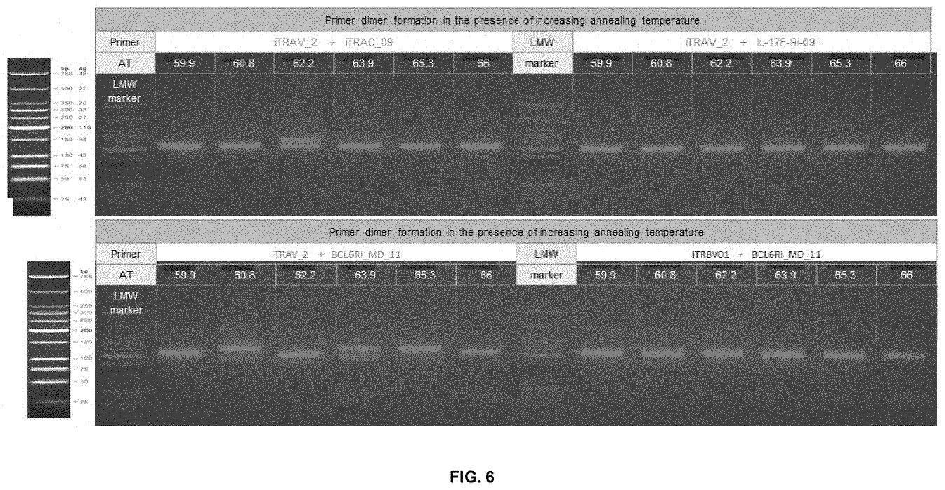

One of the most frequently attempted methods to remove primer-dimer formation is to decrease PCR cycles and to increase the annealing temperature with the concept being primer-dimer formation will be inhibited at higher temperatures. In cases of single cell amplification, high numbers of cycles are necessary to achieve amplification, because the starting copy number is low, so cycle reduction is not a viable option if sensitivity is to be maintained. In FIG. 6, Applicants demonstrate that adjusting the annealing temperature to the point at which the primers in the mix will no longer bind the target template is insufficient to remove primer-dimer formation, demonstrating that simply raising the annealing temperature will not remove the competition. Known pairs of primers that form primer-dimers were tested at various annealing temperatures. If primer-dimer production could be reduced by increasing the annealing temperature, then the primer-dimer band should have decreased in strength as the temperature was raised. Instead, in all cases, the band strength for the primer-dimer product was relatively unchanged, despite the increase in annealing temperature. There was only a slight decrease in two of the tested pairs at the highest allowable annealing temperature.

### Purposeful Spike-In of a Primer-Pair with Primer-Dimer Propensity to Compare Between arm-PCR and dam-PCR

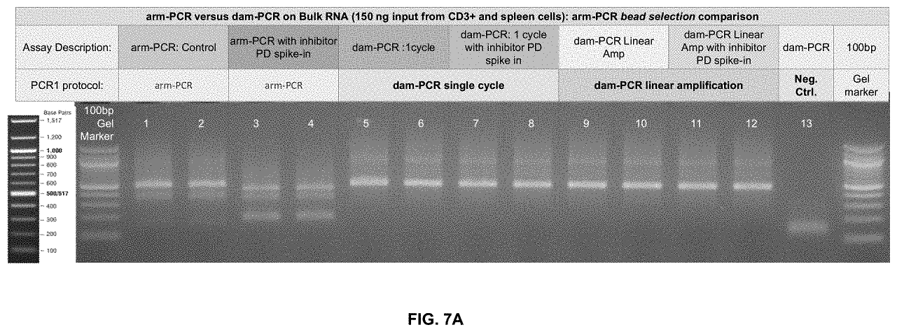

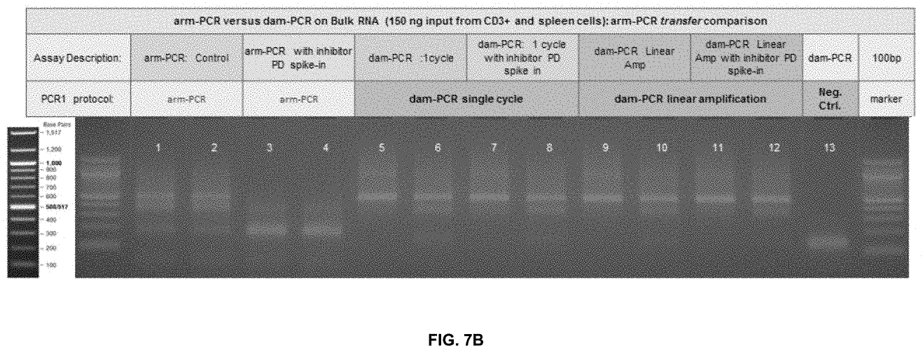

dam-PCR is capable of overcoming the inhibitory effect of primer-dimers on amplification. An arm-PCR comparison to dam-PCR experiment was performed with a multiplex mix containing 150 ng RNA from a mixture of CD3+ T-cells and spleen. The arm-PCR experiment was performed by adding a primer mix of both forward and reverse inside primers (no outside primers for better comparison) in the absence and presence of additional pairs of primers known to cause primer-dimer formation. For dam-PCR, the same reverse primer mix used in the arm-PCR amplification were added during the reverse transcription step. The first strand cDNA was selected using magnetic beads, and the second strand tagging was performed with the same forward primer mix used with the arm-PCR experiment. A comparison of single cycle dam-PCR (1 cycle of heat denaturation, annealing, and extension) and linear amplification dam-PCR (heat denaturation followed by 6-cycles of annealing and extension) was also performed. The tagged dam-PCR libraries were selected using magnetic beads, and the library was amplified with a pair of communal primers for 20 cycles. After tagging and one round of amplification with the pair of common primers, 2 μL of the dam-PCR library was used for the transfer experiment of the first PCR amplicons to a second PCR reaction (FIG. 7B) for a direct comparison to the similar transfer arm-PCR test. The remaining dam-PCR library was selected using magnetic beads and amplified for 30 more cycles with the same universal primer-pair in the presence of fresh enzyme and buffer. The total number of amplification cycles was equivalent to the arm-PCR protocol, which included an RT-PCR step of 20 cycles amplification and a second PCR of 30 cycles.

The results in FIGS. 7A and 7B clearly demonstrate that the dam-PCR strategy of primer-dimer avoidance results in highly sensitive amplification regardless of the presence of a pair of primers of high dimerization potential. Technically, with dam-PCR the offending primer pair are never in contact and, therefore, never have the opportunity to dimerize. Thus, the tagging steps are performed in two independent steps, and amplification by PCR is actually performed with a pair of common primers (instead of a multiplex mix). The only portion of primers that are multiplexed are the forward and the reverse sets (or the first primer mix and second primer mix with gDNA), respectively. Since each set, forward mix and reverse mix, are used only once, however, potential intra-mix primer-dimer pairs are never allowed to accumulate. All amplifications in FIGS. 7A and 7B include a technical replicate. FIG. 7A represents a bead selection between the first PCR reaction and the second PCR reaction, whereas FIG. 7B represents a 2 μL transfer between the first PCR reaction and the second PCR reaction. As demonstrated in FIGS. 7A and 7B, when arm-PCR is performed in the presence of a known damaging primer pair, amplification efficiency of the desired product was reduced. The effect was pronounced for the case of transfer between PCR1 and PCR2 in FIG. 7B, which eliminates the primary product band. It is clear in FIGS. 7A and 7B for the same template RNA, the dam-PCR approach results in a very strong primary product band and no primer-dimer production, particularly in FIG. 7A where there is no chance of primer carry over as with the 2 μL transfer. There is no obvious difference on the agarose gel between a single cycle and linear amplification approach to dam-PCR, indicating that single cycle tagging is a feasible approach.

### dam-PCR and Single Cell Amplification

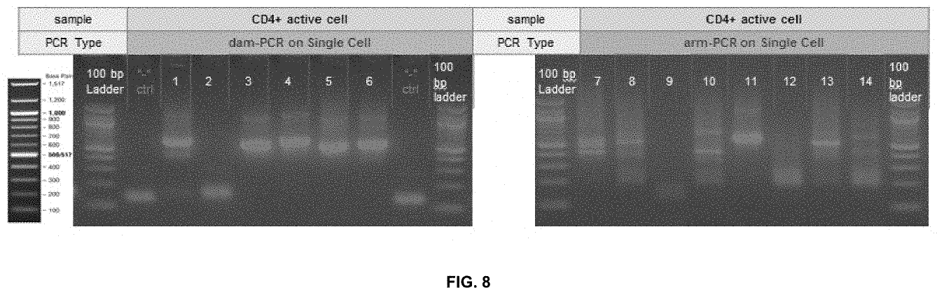

Single cells are arguably some of the most challenging templates, because the copy number of targets is low and phenotypic variation from cell to cell makes each interaction of the multiplex mix with the template dynamic. In the absence of template, certain primer pairs that do not demonstrate high propensity for primer-dimer formation in the ensemble bulk measurement can begin to dimerize due to the lack of a target. This adds a layer of complexity as the interaction of a highly multiplexed primer mix with a varying low-copy template cannot be predicted or designed around. In FIG. 8, Applicants demonstrate that at the single cell level dam-PCR produced amplicons with strong band strength that was free of primer-dimer formation, despite variation of gene expression at single cell level, whereas arm-PCR demonstrated that the dynamic nature of single cells results in varying degrees of amplification of the targets of interest with increased smearing and primer-dimer formation where primer-dimer formation was not explicitly avoided. These negative side effects reduced single cell output by resulting in dropout of targets of interest in sequencing results, necessitated increased sequencing reads be dedicated per cell to supply sufficient gene coverage, and lead to costly sequencing of non-useful primer-dimers, ultimately increasing the cost of single cell analysis significantly.

### Carryover of Primers Post-Selection

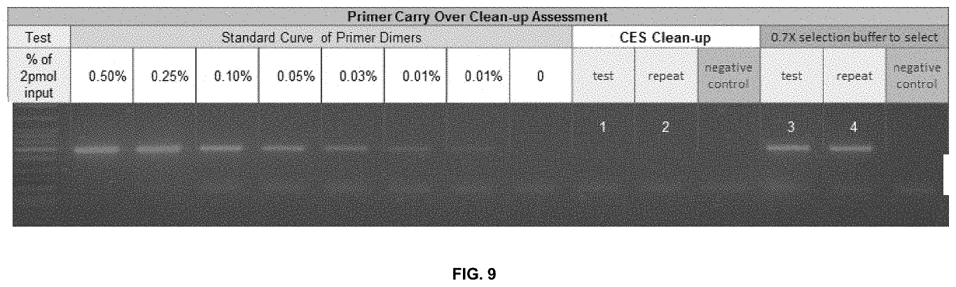

To demonstrate that the methods disclosed herein result in the nearly 100% removal of unused primer between steps, Applicants performed an experiment in which Applicants made a standard curve by serially diluting a 2 pmol stock of reverse primer from 0.5% to 0. These dilutions were used as a reference to measure the residual carryover of primer from two types of clean-up methods. The dilutions mimic cases in which a 0.5% to 0% carryover of reverse primer would be encountered between reverse transcription and second strand synthesis. A forward primer of known primer-dimer propensity was added to each of the serially diluted mixes, and the mixes were subjected to PCR to indirectly visualize the percentage of “residual” reverse primer. In the test samples, two pmols of the reverse primer were included in each of the four tests (including technical replicates). These mixes were subjected to two different methods of SPRI bead clean-up. To detect the residual primer after clean-up, 2 pmols of the same forward primer used for the serially diluted samples was added to the selected product of the test samples. In this case, the residual reverse primer after cleaning was the only source for potential amplification. The tests samples were subjected to the same PCR as the standard dilution curve. This way, the band strength of amplification for the test samples can directly indicate the percentage of residual reverse primer in the clean-up tests by comparing to the standard curve as shown in FIG. 9. The results show that the residual primer is not detectable on the gel, and therefore, had less than 0.01% carryover (or greater than 99.99% removal), for the CES selection, whereas the 0.7x SPRI selection had a 0.10% carryover (or greater than 99.9% removal).

### Application of dam-PCR to gDNA

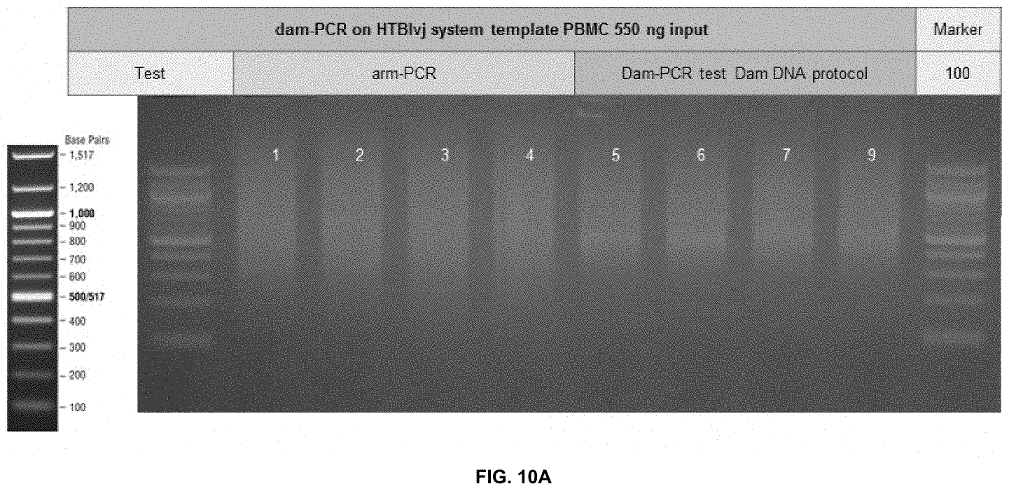

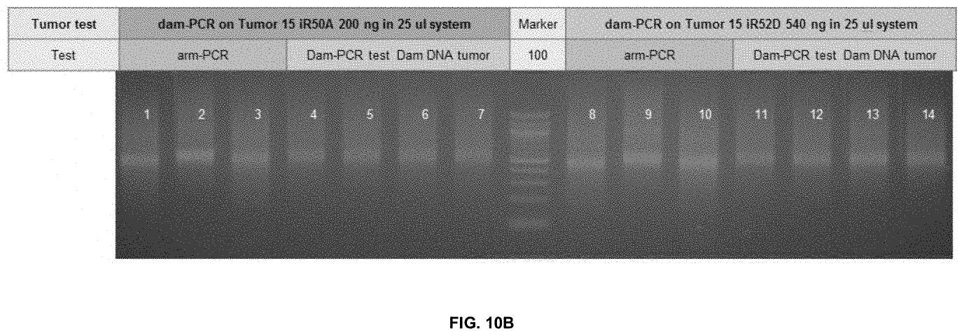

Three separate multiplex mixes covering varying targets were used to amplify gDNA with dam-PCR. One target mix covers the variable region of the human TCR beta locus with forward primers for the V-gene segment and reverse primers in the J region, which enables pick-up of the VDJ rearrangement of the TCR beta locus from gDNA. An intron gap between the rearranged variable region and C-gene together with sequencing length constraints necessitates the placement of the reverse primers in this position for gDNA coverage, requiring 13 reverse primers to cover this gene segment and over 33 forward primers. As a comparison, this mix was also used with an arm-PCR strategy. As demonstrated in FIG. 10A, dam-PCR amplification produced an amplicon band when applied to gDNA, while arm-PCR produced smearing likely due to offsite background amplifications. When forward and reverse primer mixes are used simultaneously (as with typical multiplex PCR strategies), primers can bind sites not used in the rearrangement and generate products in the first few cycles that compete with the signal of the rearrangement of interest. These off-site reactions are reduced with dam-PCR because only one cycle of tagging in either direction is allowed, and only the targets of primary interest are selected between steps.

To demonstrate that dam-PCR also works on other targets besides adaptive immune cells, a multiplex mix covering gene targets that are commonly mutated in cancerous malignancies (tumor panel; FIG. 10B) and a primer mix to detect the human leukocyte antigen type (HLA-type) of an individual were also applied to gDNA.

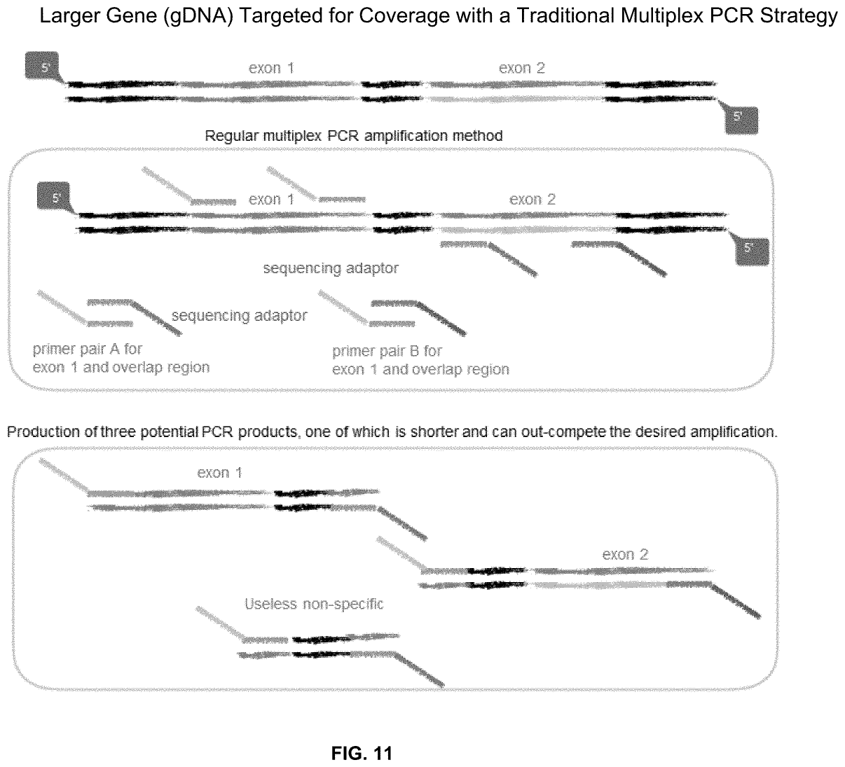

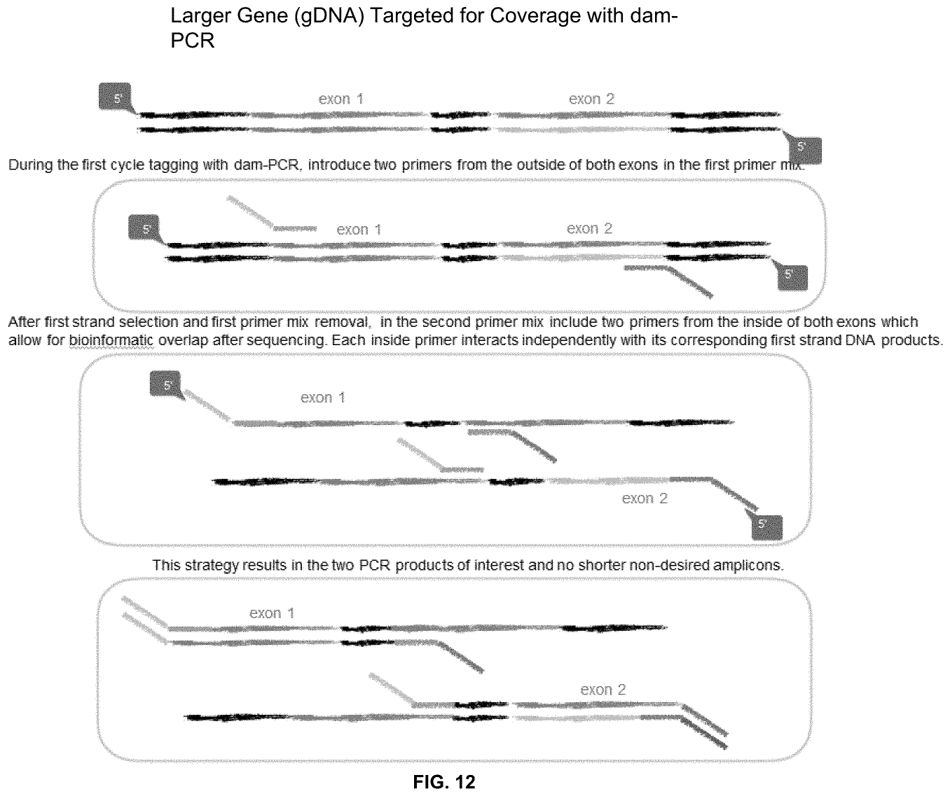

With gDNA, there is more flexibility as to which primers to include in each mix. It is not necessary to include only a forward mix or only a reverse mix. Therefore, we refer to the primer mixes as a first mix and a second mix. This strategy can be applied when designing the primer mixes to allow longer sequencing coverage by overlapping amplicon products, while reducing background, which is deleterious to both the amplification process and sequencing. The directionality of first strand cDNA synthesis with RNA requires the use of the reverse mix first. When using gDNA as a template, primer mixes do not necessarily have to be stratified into sense strand and anti-sense strand mixes. Overlapping portions can be designed with the primers facilitating easier downstream reassembly of the larger gene products bioinformatically post-sequencing. Normally, as is true of typical multiplex PCR, if the entire primer mix is allowed to interact in the same mix, off target amplifications would be produced, generating background which would compete with the product of interest, thereby producing costly background on the sequencer as shown in FIG. 11. With dam-PCR, the primer-mix strategy enables targeted sequencing with reduced background when attempting to cover larger gene targets. In the first primer mix, a pair of primers covering a much larger target can be used as illustrated in FIG. 12. Since the strands are treated independently, when the next set of primers, or second primer mix, is applied to the second strand, they will only be compatible with each respective first strand product. The interaction of the primers that can yield shorter non-useful products can now be completely avoided with the dam-PCR strategy.

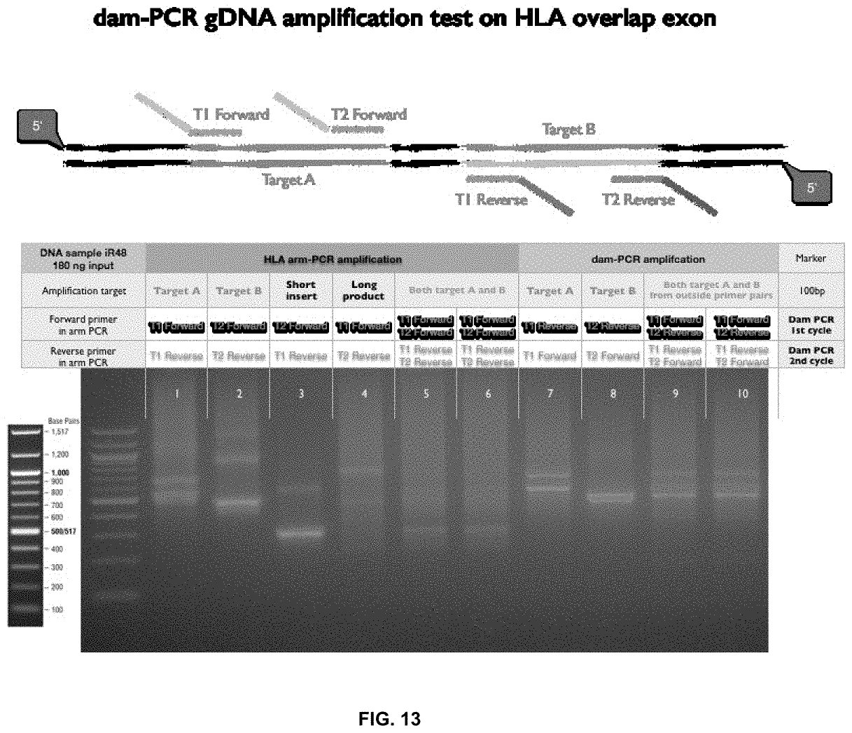

An example of both arm-PCR and dam-PCR applied to a HLA target from gDNA is provided in FIG. 13. Lanes 1 and 2 show the amplification pattern when arm-PCR was used to amplify gene Target A or Target B, respectively. Lane 3 shows the amplification pattern from the interaction of T2 Forward and T1 Reverse. This is just to demonstrate the pattern, but this product is an off-target product that can be produced when all four primers are multiplexed in the same PCR mix. Lane 4 shows the long amplification product of T1 forward primer and T2 Reverse primer. In this particular instance, this product is also less desirable because the product cannot be covered with the currently used NGS platform due to sequence length restrictions. However, NGS platforms capable of covering longer products could sequence such a product. Lanes 5-6 show the arm-PCR amplification from the fully multiplexed mix. The result is largely dominated by the less desirable short off-target product. The gene targets for Target A alone (produced from T1 Forward and T1 Reverse), Target B alone (produced from T2 forward and T2 Reverse), and Target A and B together (produced from T1 Forward and T2 Reverse) were smeared and barely present due to the short products competing with the desired product for DNA polymerase activity. Lane 7 shows the amplification pattern when dam-PCR was used to amplify gene Target A only with T1 Reverse during first cycle tagging and T1 Forward during second cycle tagging. Lane 8 shows the amplification pattern when dam-PCR was used to amplify gene Target B only with T2 Reverse during first cycle tagging and T2 Forward during second cycle tagging. Lanes 9-10 show the results from the fully multiplexed dam-PCR strategy. In the first round of tagging with the first primer mix, T1 Forward and T2 Reverse were used to generate the longer first strand products. In the second round of tagging with the second primer mix, T1 Reverse and T2 Forward interacted independently with the first strand products generated during the first strand tagging. After second strand tagging, clean-up, and amplification with a pair of primers with a tag common to the first round targets, there were no competing short products, and the amplification of the desired products was achieved. As evident in the gel image of Lanes 9-10, the product bands were the sum of the desired products represented in Lanes 1-2 or in Lanes 7-8. It is important to note that the single cycle of tagging is critical. If the primers T1 Forward and T2 Reverse were allowed to go through additional cycling like normal PCR, the short competing product could be produced. However, these potentially deleterious primers are removed in dam-PCR prior to any true amplification with the common universal primers.

References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

The various embodiments of the systems and methods described herein are exemplary. Various other embodiments for the systems and methods described herein are possible.

1. A method comprising the steps of:
a) reverse transcribing at least one first strand of cDNA from mRNA containing at least one target sequence, using a reverse primer mix, forming at least one first strand cDNA;
wherein the reverse primer mix contains at least one reverse primer configured to incorporate a reverse common primer binding site into each first strand of cDNA;
b) selecting each first strand cDNA and removing unused reverse primer;
c) synthesizing at least one second strand of cDNA from each of the at least one first strand of cDNA using a forward primer mix, forming at least one first strand:second strand complex;
wherein the forward primer mix contains at least one forward primer, each forward primer configured to bind to a particular first strand of cDNA and to incorporate a forward common primer binding site into each second strand of cDNA;
d) selecting each first strand:second strand complex and removing unused forward primer;
e) amplifying the first and second cDNA strands using a reverse common primer which binds to the at least one reverse common primer binding site and using a forward common primer which binds to the at least one forward common primer binding site; and
f) selecting the amplified cDNA strands.

2. The method of claim 1, further comprising the step of amplifying the amplified cDNA strands after step f), the amplifying comprising using a reverse common primer which binds to the at least one reverse common primer binding site and using a forward common primer which binds to the at least one forward common primer binding site.

3. The method of claim 1, wherein the reverse primer mix comprises at least one reverse primer, wherein the at least one reverse primer comprises additional nucleotides which incorporate into each first cDNA strand as an identifying marker.

4. The method of claim 1, wherein the forward primer mix comprises at least one forward primer, wherein the at least one forward primer comprises additional nucleotides which incorporate into each second cDNA strand as an identifying marker.

5. The method of claim 1, wherein each selection step b) comprises separation of cDNA strands from the reverse primer mix using magnetic beads, selection step d) comprises separation of cDNA strands from the forward primer mix using magnetic beads, and selection step f) comprises separation of cDNA strands from reverse common primer and forward common primer using magnetic beads.

6. The method of claim 1, wherein each selection step b) comprises separation of cDNA strands from the reverse primer mix by column purification, selection step d) comprises separation of cDNA strands from the forward primer mix by column purification, and selection step f) comprises separation of cDNA strands from reverse common primer and forward common primer by column purification.

7. The method of claim 1, wherein each selection step b) comprises enzymatic cleavage of the reverse primer mix, selection step d) comprises enzymatic cleavage of the forward primer mix, and selection step f) comprises enzymatic cleavage of the reverse common primer and forward common primer.

8. The method of claim 1, wherein the mRNA is obtained from a single cell.
